(19) United States
(12) Patent Application Publication
(10) Pub. No.: US 2018/0228508 A1
(43) Pub. Date: Aug. 16, 2018

(54) CUTTING TOOL WITH DISTAL END-FACE CUTTER

(71) Applicants: Itamar Harchol, Kfar Tavor (IL); Yochanan Chorev, Hod Hasharon (IL)

(72) Inventors: Itamar Harchol, Kfar Tavor (IL); Yochanan Chorev, Hod Hasharon (IL)

(21) Appl. No.: 15/431,785

(22) Filed: Feb. 14, 2017

(51) Int. Cl. A61B 17/3207; A61B 17/3201

(52) U.S. Cl. CPC A61B 17/320758; A61B 17/3201; A61B 17/3207

(57) ABSTRACT

A cutting tool includes a rotatable cutter arranged for rotation inside a hollow tube. The rotatable cutter has blades at a distal end-face thereof. Each blade has first and second cutting edges. The hollow tube includes distally-protruding scissor members in one-to-one correspondence with the blades. Each scissor member is formed with first and second inwardly facing cutting edges that face the first and second cutting edges of the rotatable cutter.

### FIELD OF THE INVENTION

The present invention relates generally to surgical cutting tools, and more specifically to a cutting tool with a distal end-face cutter, useful, for example, in arthroscopic surgical procedures.

### BACKGROUND OF THE INVENTION

Arthroscopic surgery is generally performed through two skin punctures into a joint space with direct visual inspection by an optical fiber that penetrates into the joint cavity and which is connected at the other end to a video camera. Arthroscopic surgery is done with specific small manual and electric instruments adapted to work through the skin puncture.

Not only must the skin be cut, but very often cartilage or other soft tissues must be cut, such as the meniscus in the knee or other joints. However, cartilage and other soft tissues are smooth and slidable, which can present challenges to achieve a properly placed and executed cut.

### SUMMARY OF THE INVENTION

The present invention seeks to provide an improved cutting tool with a distal end- face cutter, as is described more in detail hereinbelow. The cutting tool is particularly useful in arthroscopic surgical procedures, but may be used in other surgical procedures as well.

The cutting tool includes a rotatable cutter that is arranged for rotation inside a hollow tube. The rotatable cutter is a distal end-face cutter that has one or more blades, preferably two or more, each of which is crescent shaped. Each of the blades has a first cutting edge on its distal face, which may be crescent shaped, and a second cutting edge perpendicular to the first cutting edge, parallel to the longitudinal axis of the cutting tool.

The hollow tube includes one or more distally-protruding scissor members, to match the number of blades of the rotatable cutter (preferably two or more scissor members to match the two or more blades). Each of the scissor members is formed with first and second inwardly facing cutting edges: the first one is faces towards the first cutting edge of the blade (and may also be crescent shaped) and the second one is perpendicular to the first one, facing the second cutting edge of the blade. The second cutting edge of the scissor member may be slanted, such as at a 45° angle with respect to the longitudinal axis of the cutting tool.

The rotatable cutter is pressed against the tissue to be cut. The tissue to be cut becomes located between the cutting edges of the rotatable cutter and the cutting edges of the scissor members of the hollow tube, either by the action of pressing the cutter against the tissue or with the addition of vacuum that sucks the tissue, liquids and debris inwards between the two cutting edges (the vacuum being introduced through the hollow tube from a vacuum source). The tissue to be cut is grasped and held against the scissor members during the cutting action of the rotatable cutter, including when the rotatable cutter cuts the tissue by itself and when the rotatable cutter cuts in a scissors action by sweeping past the scissor members. The scissor members may be rotated slightly before commencing cutting in order to pinch and grasp the tissue to be cut between the scissor members and the rotatable cutter. The scissor members help the surgeon properly orient the cutting tool with respect to the tissue to be cut. The outer surface of the hollow tube may have fiduciary marks to help achieve proper orientation of the cutting tool with respect to the tissue to be cut.

Once the tissue is grasped, the rotatable cutter is rotated, either manually by rotating a rod or tube placed through the hollow tube and attached to the rotatable cutter, or by rotating with an actuator, such as a motor, connected by any suitable means to the rotatable cutter. The cutting edges of the rotatable cutter cut the tissue by themselves when they are not aligned with the scissor members. When the rotatable cutter rotates to be aligned with the scissor members, the rotatable cutter cooperates with the scissor members to cut the tissue like scissors as they rotate past the cutting edges of the scissor members, which serves as the other blade of the scissors. The rotation of the cutter in both directions also chops and grinds the tissue. The radial space between the cutting edges of the rotatable cutter and the cutting edges of the scissor members traps the tissue as it is cut. The axial space (i.e., along the longitudinal axis) between the cutting edges of the rotatable cutter and the cutting edges of the scissor members determines the depth of the cut and also aids in trapping the tissue as it is cut.

The vacuum may also be used to suck cut tissue, blood and any other debris from the cutting site.

### BRIEF DESCRIPTION OF THE DRAWINGS

The present invention will be understood and appreciated more fully from the following detailed description taken in conjunction with the drawings in which:

FIG. 1 is a simplified pictorial illustration of a cutting tool, constructed and operative in accordance with a non-limiting embodiment of the present invention;

FIG. 2 is a simplified exploded illustration of the cutting tool;

FIG. 3 is a simplified end-view illustration of the cutting tool;

FIG. 4 is a simplified cutaway view illustration of the first cutting edges of the rotatable cutter acting as scissors in cooperation with the first cutting edges of the scissor members;

FIG. 5 is a simplified cutaway view illustration of the second cutting edges of the rotatable cutter acting as scissors in cooperation with the second cutting edges of the scissor members;

FIG. 6 is a simplified perspective illustration of the cutting tool assembly with actuator and vacuum source, in accordance with a non-limiting embodiment of the present invention; and

FIGS. 7A and 7B are simplified pictorial illustrations of the cutting tool with a bridge element between scissor members of the cutting tool, in accordance with a non-limiting embodiment of the present invention.

### DETAILED DESCRIPTION OF EMBODIMENTS

Reference is now made to FIGS. 1-3, which illustrate a cutting tool 10, constructed and operative in accordance with a non-limiting embodiment of the present invention.

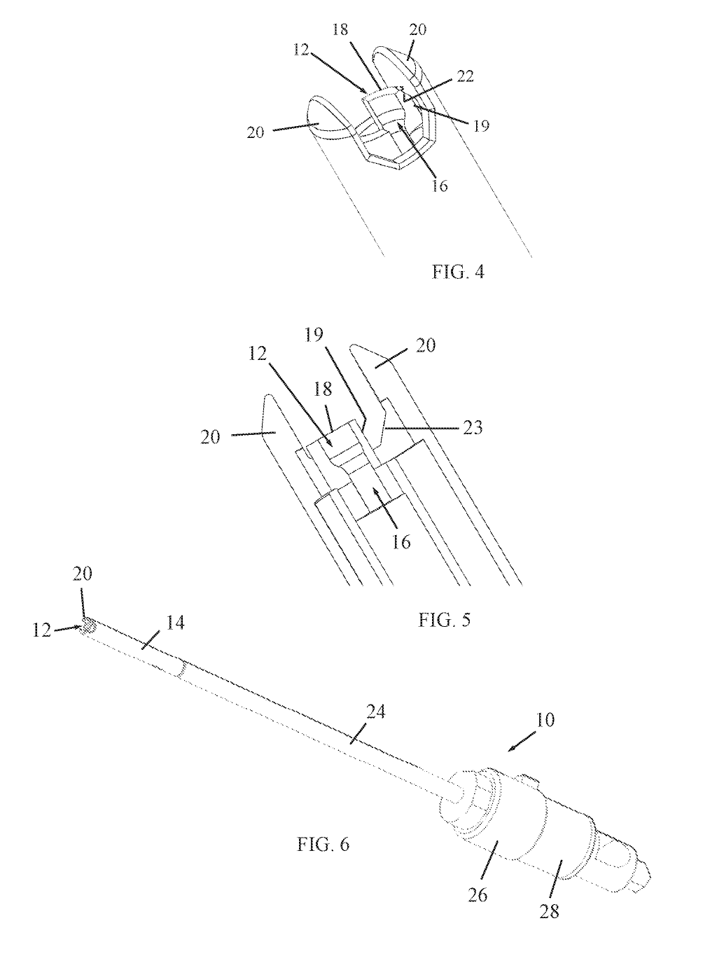

Cutting tool 10 includes a rotatable cutter 12 that is arranged for rotation inside a hollow tube 14. The rotatable cutter 12 is a distal end-face cutter, which may be disc-shaped or other shapes. The rotatable cutter 12 has one or more blades 16, preferably two or more, each of which may be crescent shaped. Each of the blades 16 has a first cutting edge 18 on its distal face, which may be beveled and crescent shaped, and a second cutting edge 19 perpendicular to the first cutting edge 18, parallel to the longitudinal axis of the cutting tool 10. The first and second cutting edges 18 and 19 are seen clearly in FIGS. 4 and 5.

The hollow tube 14 includes one or more distally-protruding scissor members 20, to match (in one-to-one correspondence with) the number of blades 16 of the rotatable cutter 12. Thus, there are preferably two or more scissor members 20 to match the two or more blades 16. Each of the scissor members 20 is formed with first and second inwardly facing cutting edges 22 and 23, respectively. The first cutting edge 22 faces towards the first cutting edge 18 of the blade 16 (and may also be crescent shaped). The second cutting edge 23 is perpendicular to the first cutting edge 22, and faces the second cutting edge 19 of the blade 16. The first and second cutting edges 22 and 23 are seen clearly in FIGS. 4 and 5. As seen in FIG. 5, the second cutting edge 23 of the scissor member may be slanted, such as at a 45° angle with respect to the longitudinal axis of the cutting tool.

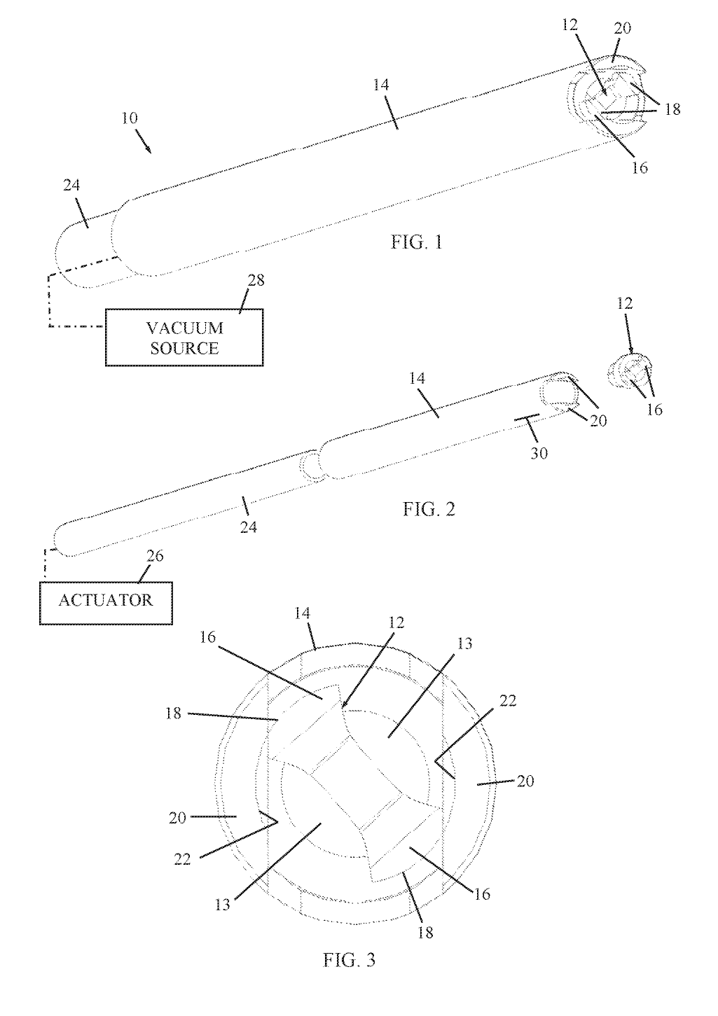

The rotatable cutter 12 may be rotated manually by rotating a manipulator rod or tube 24 (also called an actuator 24) placed through the hollow tube 14 and attached to the rotatable cutter 12. The proximal end of manipulator tube 24 may have a handle or knob (not shown) to assist in grasping and turning the manipulator tube 24. Alternatively, rotatable cutter 12 may be rotated with an actuator 26 (seen in FIG. 6, and also shown schematically in broken lines in FIG. 2), such as a motor, connected by any suitable means to the rotatable cutter 12.

A vacuum source 28 (seen in FIG. 6, and also shown schematically in broken lines in FIG. 1) may be provided which is in fluid communication with the lumen of hollow tube 14 (such as by a suitable fluidly sealed connector). The vacuum from vacuum source 28 may be used to suck in tissue between the rotatable cutter 12 and the scissor members 20. The vacuum may also be used to suck cut tissue, blood and any other debris from the cutting site.

In use, the rotatable cutter 12 may be pressed against the tissue to be cut. The tissue to be cut becomes located between the cutting edges of the rotatable cutter 12 and the cutting edges of the scissor members 20, either by the action of pressing the cutter 10 against the tissue or with the addition of the vacuum that sucks the tissue inwards between the corresponding cutting edges of the rotatable cutter and scissor members. (Some of the areas for sucking the tissue are indicated by reference numeral 13 in FIG. 3.) The scissor members 20 may be rotated slightly before commencing cutting in order to pinch and grasp the tissue to be cut between the scissor members 20 and the rotatable cutter 12. The scissor members 20 help the surgeon properly orient the cutting tool 10 with respect to the tissue to be cut. The outer (or other suitable) surface of the hollow tube 14 may have one or more fiduciary marks 30 (FIG. 2) to help achieve proper orientation of the cutting tool 10 with respect to the tissue to be cut.

The tissue to be cut is grasped and held against the scissor members 20 during the cutting action of the rotatable cutter 12, including when the rotatable cutter 12 cuts the tissue by itself and when the rotatable cutter 12 cuts in a scissors action by sweeping past the scissor members 20.

Once the tissue is grasped, the rotatable cutter 12 is rotated, either manually or with actuator 26. The cutting edges 18 of the rotatable cutter 12 cut the tissue by themselves when they are not aligned with the scissor members 20. When the rotatable cutter 12 rotates to be aligned with the scissor members 20, the rotatable cutter 12 cooperates with the scissor members 20 to cut the tissue like scissors as they rotate past the cutting edges of the scissor members 20, which serves as the other blade of the scissors, as is now described with further reference to FIGS. 4 and 5.

In FIG. 4, the first cutting edges 18 of the blade 16 of rotatable cutter 12 act as scissors in cooperation with the first cutting edges 22 of the scissor members 20. The first cutting edges 22 of the scissor members 20 slightly overlap the first cutting edges 18 of the rotatable cutter 12.

In FIG. 5, the second cutting edges 19 of the blade 16 of rotatable cutter 12 act as scissors in cooperation with the second cutting edges 23 of the scissor members 20. The second cutting edges 19 gradually slice the tissue as they rotate past the ramp (inclined surface) of the second cutting edges 23 of the scissor members 20.

The cutting tool may first cut the tissue with the first cutting edges of the rotatable cutter 12 and of the scissor members 20, and then with their second cutting edges. Alternatively, the cutting tool may first cut the tissue with the second cutting edges of the rotatable cutter 12 and of the scissor members 20, and then with their first cutting edges.

The rotatable cutter 12 cuts as a dual-action cutter: it cuts by itself as a distal end-face cutter and it cuts as scissors in cooperation with the scissor members 20. The scissor members 20 are also dual-action members: they serve as the other blade of the scissors for cutting and they also serve to grasp the tissue being cut.

The scissor members 20 can pinch or trap the tissue which is to be cut (e.g., rotating or tilting the scissor members pinches or grabs the tissue). The axial space (i.e., along the longitudinal axis of cutter 10) between the cutting edges of the rotatable cutter 12 and the cutting edges of the scissor members 20 determines the depth of the cut and also aids in trapping the tissue as it is cut.

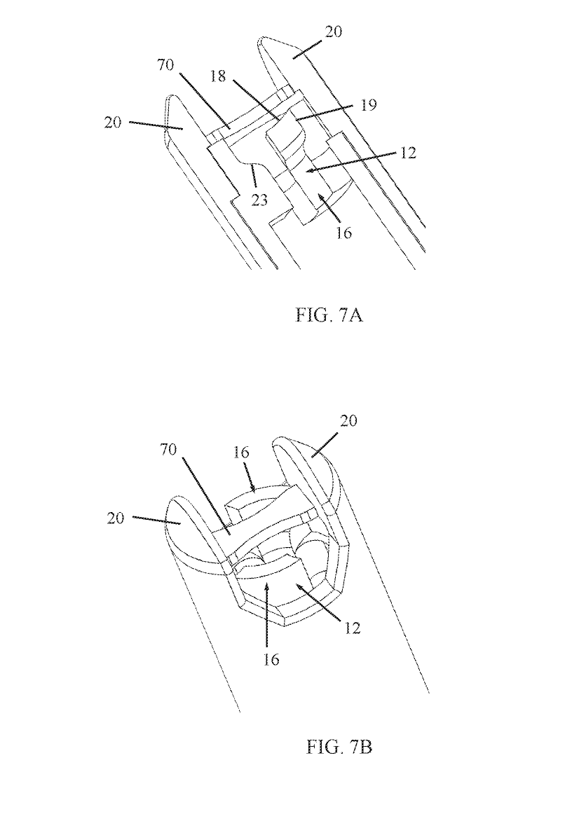

Reference is now made to FIGS. 7A and 7B, which illustrate the cutting tool with a bridge element 70 extending between and connected to scissor members 20, in accordance with a non-limiting embodiment of the present invention.

Bridge element 70 may be used to help stiffen the scissor members 20 so they do not vibrate during the cutting action, thereby achieving a more precise and controlled cut. In another alternative embodiment, one scissor member 20 may be used which extends a full 360° around the end of the cutting tool or close to 360°. Such a structure also eliminates any vibration but may hinder in the suction of liquids and debris.

What is claimed is:

1. A cutting tool comprising:
a rotatable cutter arranged for rotation inside a hollow tube, said rotatable cutter comprising one or more blades at a distal end-face thereof, each of said blades comprising a first cutting edge on said distal end-face and a second cutting edge perpendicular to said first cutting edge; and
wherein said hollow tube comprises one or more distally-protruding scissor members in one-to-one correspondence with said one or more blades, each of said scissor members being formed with first and second inwardly facing cutting edges that face the first and second cutting edges of said rotatable cutter.

2. The cutting tool according to claim 1, wherein said one or more blades of said rotatable cutter are configured to cut as a distal end-face cutter when said one or more blades are not aligned with said one or more scissor members, and are configured to cut as a scissors in cooperation with said one or more scissor members when said one or more blades are rotated to be aligned with said one or more scissor members.

3. The cutting tool according to claim 1, further comprising an actuator attached to said rotatable cutter for rotating said rotatable cutter.

4. The cutting tool according to claim 3, wherein said actuator comprises a manipulator tube placed through said hollow tube and attached to said rotatable cutter.

5. The cutting tool according to claim 3, wherein said actuator comprises a motor.

6. The cutting tool according to claim 1, wherein said first cutting edges of said rotatable cutter are crescent shaped.

7. The cutting tool according to claim 1, further comprising a vacuum source in fluid communication with a lumen of said hollow tube.

8. The cutting tool according to claim 1, wherein said hollow tube comprises one or more fiduciary marks.

9. The cutting tool according to claim 1, wherein said one or more scissor members are configured to pinch or trap tissue being cut.

10. The cutting tool according to claim 1, wherein an axial space between said cutting edges of said rotatable cutter and said cutting edges of said one or more scissor members determines a depth of cut of said cutting tool.

11. The cutting tool according to claim 1, further comprising a bridge element extending between and connected to said one or more scissor members.
